(19) United States
(12) Patent Application Publication
(10) Pub. No.: US 2022/0061431 A1
(43) Pub. Date: Mar. 3, 2022

(54) Disinfecting Gloves

(71) Applicant: Michael Krueger, Superior, WI (US)

(72) Inventor: Michael Krueger, Superior, WI (US)

(21) Appl. No.: 17/009,772

(22) Filed: Sep. 1, 2020

(51) Int. Cl. A41D 19/00; A61L 2/18

(52) U.S. Cl. CPC A41D 19/0082; A41D 2500/50; A61L 2/18; A41D 19/0058

(57) ABSTRACT

The sanitizing glove is built to not only provide the user with protection from external germs, but to sanitize the touched surfaces by use. On the front of the glove where the wearer's palm and fingers are behind the glove is attached a sanitizing surface akin to a disinfectant wipe. As the wearer of the glove interacts with objects such as handles, knobs, or buttons, the sanitizing liquid of the glove's front cleans the objects touched. This procedure protects not only the glove wearer from germs, but anyone else who interacts with the same objects soon after. Over time, the sanitizing liquid on the front of the glove may begin to dry or run down. It can be refreshed by use of a sanitizing spray.

### BACKGROUND

A wet wipe, also known as a wet towel or a moist towelette, or a baby wipe in specific circumstances, is a small moistened piece of plastic or cloth that often comes folded and individually wrapped for convenience. Wet wipes are used for cleaning purposes like personal hygiene and household cleaning. Cleansing pads for preventing infection are usually saturated with alcohol and bundled in sterile packages. Hands and instruments may be disinfected with these pads while treating wounds. Disinfecting cleansing pads are often included in first aid kits for this purpose. There have been no products available as original equipment or as an aftermarket to address this problem.

Medical gloves are disposable gloves used during medical examinations and procedures to help prevent cross-contamination between caregivers and patients. Medical gloves are made of different polymers including latex, nitrile rubber, polyvinyl chloride and neoprene; they come unpowdered, or powdered with corn starch to lubricate the gloves, making them easier to put on the hands. Besides medicine, medical gloves are widely used in chemical and biochemical laboratories. Medical gloves offer some basic protection against corrosives and surface contamination. There have been no products available as original equipment or as an aftermarket to address this problem either.

There exists a need for a disinfecting glove that is not being met by any known or disclosed device or system of present.

### SUMMARY OF THE INVENTION

The sanitizing glove is built to not only provide the user with protection from external germs, but to sanitize the touched surfaces by use. On the front of the glove where the wearer's palm and fingers are behind the glove is attached a sanitizing surface akin to a disinfectant wipe. As the wearer of the glove interacts with objects such as handles, knobs, or buttons, the sanitizing liquid of the glove's front cleans the objects touched. This procedure protects not only the glove wearer from germs, but anyone else who interacts with the same objects soon after. Over time, the sanitizing liquid on the front of the glove may begin to dry or run down. It can be refreshed by use of a sanitizing spray.

### BRIEF DESCRIPTION OF THE DRAWINGS

FIG. 1 is a front-facing view of the disinfecting glove in accordance with an embodiment of the present disclosure.

FIG. 2 is a cross-section of the disinfecting and protective layers of the disinfecting glove in accordance with an embodiment of the present disclosure.

FIG. 3 is a cross section of the first, second and third layers of the disinfecting glove in accordance with an embodiment of the present disclosure.

FIG. 4 is a depiction of second layer finger and thumb portions discrete from a palm portion in accordance with an embodiment of the present disclosure.

FIG. 5 is a depiction of three layers having continuous finger, thumb and palm portions in accordance with an embodiment of the present disclosure.

FIG. 6 is a depiction of three layers having a discrete finger, thumb and palm portions for one layer in accordance with an embodiment of the present disclosure.

FIG. 7 is a flow chart for a method of disinfecting via a disinfecting glove in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Throughout the present disclosure the term [term] is used to refer to [describe, rest of sentence]. The term [term] refers to [describe, rest of sentence].

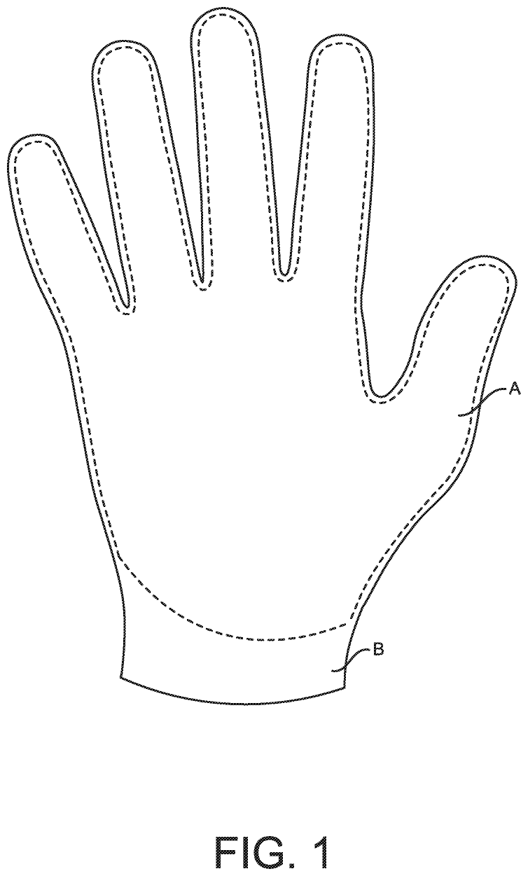

FIG. 1 is a front-facing view of the disinfecting glove in accordance with an embodiment of the present disclosure. The glove B functions as a medical glove and additionally has a sanitizing surface A attached to the palm and front of the glove so the wearer can clean the objects interacted with by regular use. The finger, thumb and palm portions of the glove are continuous in this depiction.

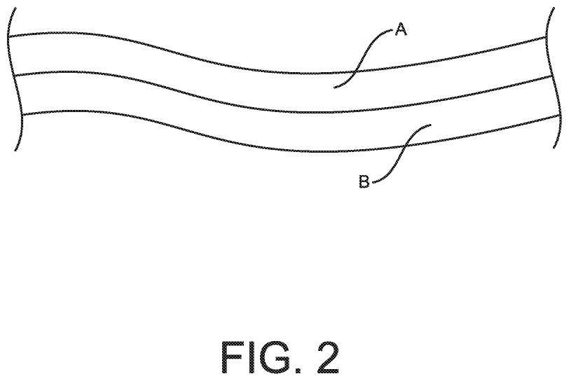

FIG. 2 is a cross-section of the disinfecting and protective layers of the disinfecting glove in accordance with an embodiment of the present disclosure. The depiction includes a glove layer B and a disinfecting layer A taken through a finger section of the disinfecting glove.

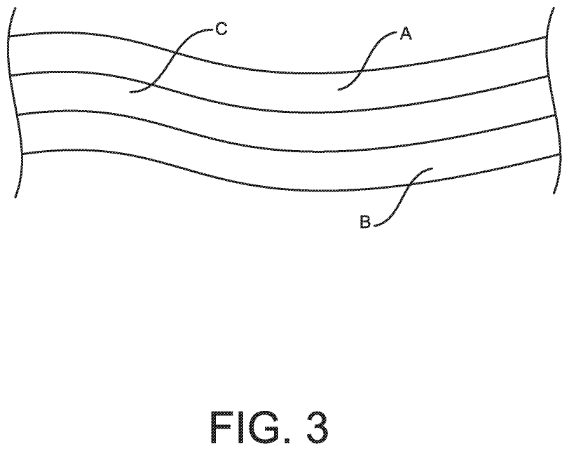

FIG. 3 is a cross section of the first, second and third layers of the disinfecting glove in accordance with an embodiment of the present disclosure. The depiction includes a glove layer B, a disinfecting layer A and a third layer C taken through a finger section of the disinfecting glove. In some embodiments, the third layer C is one of the top layer and the bottom layer in reference to the hand of the user.

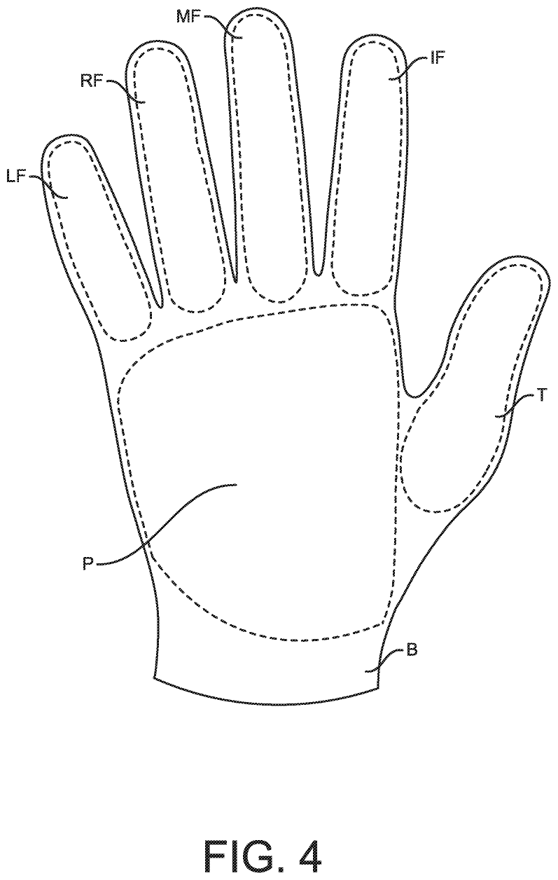

FIG. 4 is a depiction of second layer finger and thumb portions discrete from a palm portion in accordance with an embodiment of the present disclosure. The depiction includes the glove layer B, the thumb portion T on another layer, the Index Finger IF portion on the other layer, the Middle Finger portion MF on the other layer, the Ring Finger RF portion on the other layer, the Little Finger LF portion on the other layer and the palm portion P on the other layer. The 5 phalangeal portions and the palm portion are discrete in some embodiments and contiguous in other embodiments.

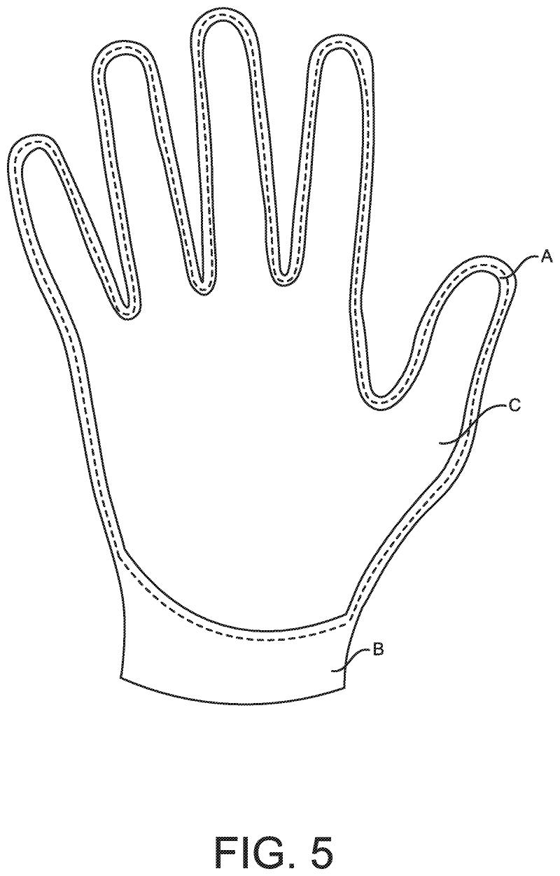

FIG. 5 is a depiction of three layers having continuous finger, thumb and palm portions in accordance with an embodiment of the present disclosure. The depiction includes continuous phalangeal and palm portions for all three layers including the glove layer B, the second layer A and the third layer C. Any one of these layers may contain antiseptic and disinfectant properties, absorbent properties and scrubbing properties according to medical needs and manufacturing constraints.

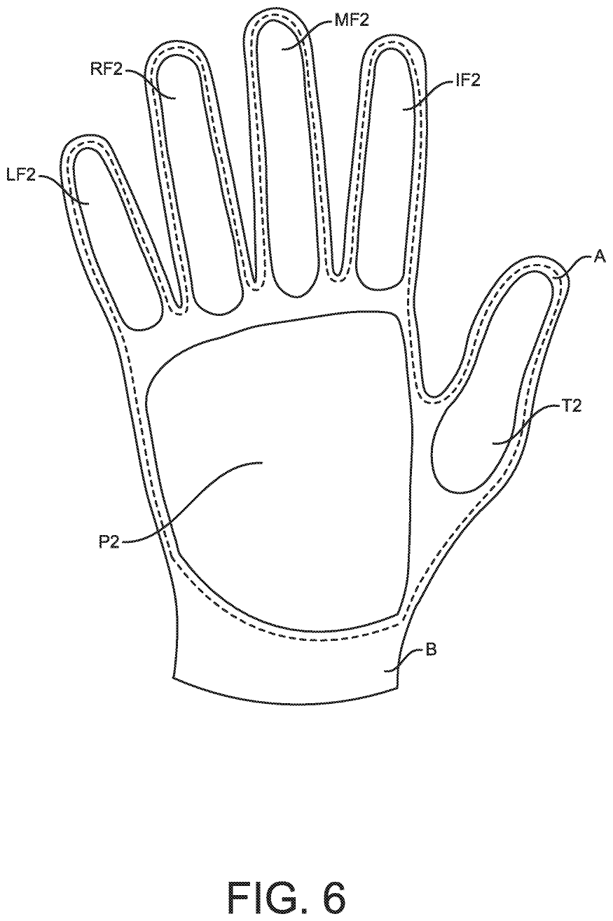

FIG. 6 is a depiction of three layers having a discrete finger, thumb and palm portions for one layer in accordance with an embodiment of the present disclosure. The depiction includes a glove layer B, a second layer A and a third layer comprising a thumb portion T2, a Index Finger portion IF2, a Middle Finger portion MF2, and Ring Finger portion RF2 and a Little Finger portion LF2 either as an intermediate layer to layers 1 and 2 or as a top or bottom layer in respect to the user's hand therein. Any one of these first, second and third layers may contain antiseptic and disinfectant properties, absorbent properties and scrubbing properties according to medical needs and manufacturing constraints.

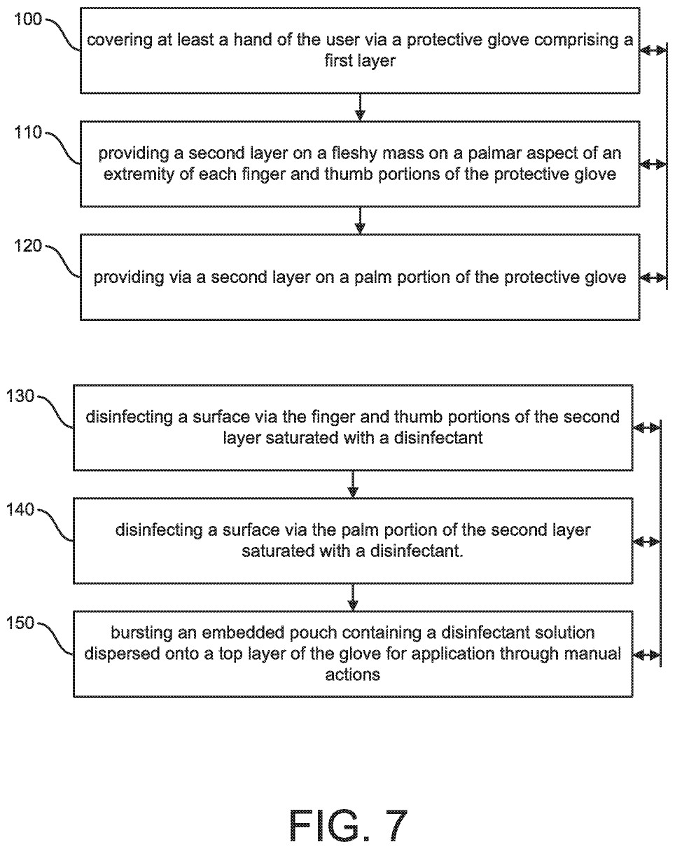

FIG. 7 is a flow chart for a method of disinfecting via a disinfecting glove in accordance with an embodiment of the present disclosure. The method of disinfecting via a hand of a user includes covering 100 at least a hand of the user via a protective glove comprising a first layer. The method also includes providing 110 a second layer on a fleshy mass on a palmar aspect of an extremity of each finger and thumb portions of the protective glove. The method additionally includes providing 120 via a second layer on a palm portion of the protective glove.

Embodiments of the method include disinfecting 130 a surface via the finger and thumb portions of the second layer saturated with a disinfectant. Embodiments further include disinfecting 140 a surface via the palm portion of the second layer saturated with a disinfectant. Embodiments yet include bursting 150 an embedded pouch containing a disinfectant solution dispersed onto a top layer of the glove for application through manual actions.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A glove for a hand of a user, the glove comprising:
a protective glove comprising a first layer configured to cover at least the hand of the user;
a second layer on a fleshy mass on a palmar aspect of an extremity of each finger and thumb portions of the protective glove; and
a second layer on a palm portion of the protective glove.

2. The glove of claim 1, wherein the protective glove comprises a polymer including latex, nitrile rubber, polyvinyl chloride and neoprene and composites thereof.

3. The glove of claim 1, wherein the finger and thumb portions and the palm portion are discrete portions in a same secondary layer of the protective glove and contain a disinfectant.

4. The glove of claim 1, wherein the finger and thumb portions and the palm portion are contiguous portions in a same secondary layer of the protective glove and contain a disinfectant.

5. The glove of claim 1, wherein the finger and thumb portions and the palm portion are a continuous portion.

6. The glove of claim 1, wherein each second layer portion comprises a disinfectant absorbent material which is initially dry.

7. The glove of claim 1, wherein each second layer is presaturated with a disinfectant solution.

8. The glove of claim 1, wherein the finger and thumb portions are presaturated with a disinfectant solution and the palm portion is dry and absorbent to the disinfectant solution.

9. The glove of claim 1, wherein the palm portion is presaturated with a disinfectant solution and the finger and thumb portions are dry and absorbent to the disinfectant solution.

10. The glove of claim 1, further comprising an intermediate layer disposed between the second layer of the protective glove first layer, the intermediate layer comprising a burstable pouch of disinfectant.

11. The glove of claim 1, further comprising an intermediate layer disposed between the second layer of the protective glove first layer, the intermediate layer absorbent to a disinfectant.

12. The glove of claim 1, wherein the second layer portions comprise an dermabrasive material.

13. The glove of claim 1, further comprising an intermediate layer disposed between the second layer of the protective glove first layer, wherein the second layer is dermabrasive and the intermediate layer is disinfecting.

14. The glove of claim 1, further comprising an intermediate layer disposed between the second layer and the protective glove first layer, wherein the finger and thumb portions and the palm portion are discrete.

15. The glove of claim 1, further comprising an intermediate layer disposed between the second layer and the protective glove first layer, wherein the finger and thumb portions and the palm portion are contiguous.

16. The glove of claim 1, further comprising an intermediate layer disposed between the second layer and the protective glove first layer, wherein the finger and thumb portions and the palm portion are continuous.

17. A glove for a hand of a user, the glove comprising:
a protective glove comprising a first layer configured to cover at least the hand of the user;
a second layer on a fleshy mass on a palmar aspect of an extremity of each finger and thumb portions of the protective glove;
a second layer on a palm portion of the protective glove; and
an intermediate layer on the finger and thumb and palm portions between the first and the second layer of the protective glove.

18. A method of disinfecting via a hand of a user, the method comprising:
covering at least a hand of the user via a protective glove comprising a first layer;
providing a second layer on a fleshy mass on a palmar aspect of an extremity of each finger and thumb portions of the protective glove; and
providing via a second layer on a palm portion of the protective glove.

19. The method of claim 18, further comprising disinfecting a surface via the finger and thumb portions of the second layer saturated with a disinfectant.

20. The method of claim 18, further comprising disinfecting a surface via the palm portion of the second layer saturated with a disinfectant.
